(19) United States
(12) Patent Application Publication
(10) Pub. No.: US 2026/0103450 A1
(43) Pub. Date: Apr. 16, 2026

(54) METHOD FOR MAKING HALOMETHYLFURFURAL PRODUCTS USING PRETREATED BIOMASS

(71) Applicant: Origin Materials Operating, Inc., West Sacramento, CA (US)

(72) Inventors: Amber K. PHILLIPS, Sacramento, CA (US); Jonathan C. OVERTON, Fond du Lac, WI (US); Evgeny BELETSKIY, Sacramento, CA (US); Petar T. LACHKOV, Sacramento, CA (US); David P. BALLOW, Sugar Land, TX (US)

(21) Appl. No.: 19/421,773

(22) Filed: Dec. 16, 2025

Related U.S. Application Data
(63) Continuation of application No. PCT/US24/36002, filed on Jun. 28, 2024.
(60) Provisional application No. 63/524,135, filed on Jun. 29, 2023.

(51) Int. Cl. C07D 307/46

(52) U.S. Cl. CPC C07D 307/46

(57) ABSTRACT

The present disclosure describes a method used to make halomethylfurfural products by using pretreated biomass. The method uses a biomass pretreatment process to produce pulp products that are processed to increase the yield of halomethylfurfural products and decrease the yield of solid carbonaceous by-products. In some aspects of the present disclosure, a pulp product that is substantially free of hemicellulose (and/or C5 sugars therein) and/or lignin is produced.

### CROSS REFERENCE TO RELATED APPLICATION

This application is a continuation of International Application No. PCT/US2024/036002, filed Jun. 28, 2024, which claims the benefit of and priority to the earlier filing date of U.S. Provisional Application No. 63/524,135, filed on Jun. 29, 2023, the entirety of each is incorporated herein by reference.

### FIELD

Disclosed herein is a method for making halomethylfurfural compounds using a pulp product obtained from a pretreated biomass, along with compositions obtained from aspects of the method.

### BACKGROUND

Substitutes to petroleum derivatives used in transportation, energy, and raw materials for industrial activities need to be developed to continue to reduce the dependence on fossil fuels. For example, fossil fuel-derived polymers are used to produce containers, adhesives, and toys; and fine chemicals are used for to make “everyday life” products, such as pharmaceuticals, detergents, and cosmetics. Biomass is a potential substitute for petroleum derivatives because it can be utilized to produce biofuels, biopolymers, high-value products, fine chemicals, and also can serve as a power source.

Cellulose from biomass can be used to produce furan-based derivatives, which can be used in other chemical reactions or downstream bioprocesses to produce biofuels, fuel additives, or plastics. For example, 5-(chloromethyl)furfural (or “CMF”) is a promising diesel fuel additive and can also be converted into 5-methylfurfural, which is a promising biofuel candidate. However, most methods for making CMF have associated drawbacks that limit their industrial applicability.

### SUMMARY

A method according to aspects of the disclosure is described. In some aspects, the method comprises: combining (i) a pulp product obtained from pretreating a biomass feedstock with (ii) a first reaction mixture comprising, a mineral acid, water, and an organic solvent to form a second reaction mixture; and heating the second reaction mixture a reaction temperature ranging from 30° C. to 300° C. to produce a halomethylfurfural product-containing composition; and isolating the halomethylfurfural product from the halomethylfurfural product-containing composition.

In some aspects, the method comprises: performing a pretreatment process on a biomass feedstock to produce a pulp product that is substantially free of hemicellulose (including C5 sugars therein) and/or lignin; exposing the pulp product to a reaction mixture comprising, a mineral acid, water, and an organic solvent at a reaction temperature ranging from 30° C. to 300° C. to produce a halomethylfurfural product-containing composition; and isolating the halomethylfurfural product from the halomethylfurfural product-containing composition.

In some aspects, the method comprises: performing a pretreatment process on a biomass feedstock, wherein the pretreatment process comprises exposing a mixture comprising the biomass feedstock to a protic solvent and acetone, adding a ballast gas at a pressure ranging from 0 psig to 1000 psig, and heating the mixture at a temperature ranging from 120° C. to 250° C. to produce a pulp product that is substantially free of hemicellulose (including C5 sugars therein) and/or lignin; exposing the pulp product to a reaction mixture comprising hydrochloric acid, water, and toluene at a reaction temperature ranging from 100° C. to 175° C. to produce a 5-(chloromethyl)furfural product-containing composition; and isolating 5-(chloromethyl)furfural from the 5-(chloromethyl)furfural product-containing composition; wherein any water present during the pretreatment process is present at an amount below 10 wt. %.

Also disclosed is a composition, comprising a halomethylfurfural product made according to any or all of the above method aspects.

The foregoing and other objects, features, and advantages of the invention will become more apparent from the following detailed description, which proceeds with reference to the accompanying figures.

### BRIEF DESCRIPTION OF THE DRAWINGS

FIG. 1 is a graph showing CMF yield (wt %) as a function of time for certain pretreated pulp products that were exposed to an HMF production process according to aspects of the present disclosure.

FIG. 2 is a graph showing values for CMF-to-solid carbonaceous by-product material (SCM) ratios and CMF-to-total by-product ratios obtained after performing certain pretreatments in combination with a halomethylfurfural (referred to herein as “HMF”) production process according to aspects of the present disclosure.

### DETAILED DESCRIPTION

### Overview of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements unless the context clearly indicates otherwise.

The methods described herein should not be construed as limiting in any way. Instead, the present disclosure is directed toward all novel and non-obvious features and aspects of the present disclosure, alone and in various combinations and sub-combinations with one another. The disclosed methods are not limited to any specific aspect or feature or combinations thereof, nor do the disclosed methods require that any one or more specific advantages be present or problems be solved. Any theories of operation are to facilitate explanation, but the methods are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed devices and methods can be used in conjunction with other devices and methods. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art. Furthermore, examples may be described with reference to directions indicated as “above,” “below,” “upper,” “lower,” and the like. These terms are used for convenient description, but do not imply any particular spatial orientation unless so indicated.

In some examples, values, procedures, or devices may be referred to as “lowest,” “best,” “minimum,” or the like. It will be appreciated that such descriptions are intended to indicate that a selection among many used functional alternatives can be made, and such selections need not be better, smaller, or otherwise preferable to other selections.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

The following abbreviations and terms and definitions are provided:

HMF: halomethylfurfural, wherein “halo” represents a halogen.

CMF: 5-(chloromethyl)furfural.

SCM: Solid carbonaceous by-product material.

Alkyl: A monoradical saturated hydrocarbon chain. The length of the alkyl chain may vary. In certain embodiments, the alkyl chain may be 1 to 20 carbon atoms (e.g., C1-20 alkyl). In one embodiment, the alkyl chain may be 4 to 15 carbons (e.g., C4-15 alkyl), or 10 to 13 carbons (e.g., C10-13 alkyl). The alkyl chain may be linear or branched. Linear alkyl chains may include, for example, n-propyl, n-butyl, n-hexyl, n-heptyl, n-octyl, n-nonanyl, n-decyl, n-undecyl, and n-dodecyl. Branched alkyl chains may include, for example, isopropyl, sec-butyl, isobutyl, tert-butyl, and neopentyl.

Biomass: Plant material made up of organic compounds biomolecules, which may include proteins, lipids, carbohydrates, lignin, clays, and or inorganic salts, and the like. Biomass may originate from various sources. For example, biomass may originate from agricultural materials (e.g., corn stover, rice hulls, peanut hulls, spent grains), processing waste (e.g., paper sludge), and recycled cellulosic materials (e.g., cardboard, old corrugated containers (OCC), old newspaper (ONP), or mixed paper). Other examples of suitable biomass may include wheat straw, paper mill effluent, newsprint, municipal solid wastes, wood chips, forest thinings, slash, miscanthus, switchgrass, sorghum, bagasse, manure, wastewater biosolids, green waste, and food/feed processing residues. Lignocellulosic is the most abundant type of biomass and can be found as part of the cell walls of plants (e.g., wheat, barley, rice straws, corn cobs, and the like). Lignocellulosic biomass can be made up of cellulose, hemicellulose, and/or lignin.

Five-Carbon Sugars (or “C5 sugars”): Sugars having a monomeric unit of 5 carbons. Such sugars may exist in monomeric, dimeric, or polymeric form. Some C5 sugars can be present in hemicellulose, which can be found in cellulosic and lignocellulosic biomass. In some aspects of the present disclosure, C5 sugars can include, but are not limited to, xylose, arabinose, and/or methylated sugars (e.g., methyl-pentopyranoside, methyl-D-xylopyranoside, or methyl 3-O-acetylpentopyranoside), and the like.

Pretreatment Process: A process that can involve physical, chemical, physiochemical, and/or biological methods for treating biomass to selectively isolate one or more components within the biomass. For example, a pretreatment process can be used to selectively isolate one or more components from biomass, such as the cellulose, hemicellulose, and/or lignin content therein. Physical pretreatments can include the application of mechanical means and application of electromagnetic or sound waves to rupture the components of biomass. Chemical pretreatments can include the application of acids, bases, or solvents for the dissolution of the different biomass components, to facilitate accessing components such as cellulose and/or hemicellulose for downstream processing. Physicochemical pretreatments can include hydrothermal/liquid hot water treatments, steam explosion, and/or such methods combined with physical and chemical pretreatments. Biological pretreatments utilize enzymes and microorganisms to degrade biomass such as cellulolytic enzymes, ligninolytic enzymes, hemicellulolytic enzymes and lytic polysaccharide monooxygenases, which degrade selectively the substrate to convert it into saccharides and other valuable products. In particular aspects of the disclosure, the pretreatment process involves using a chemical pretreatment according to aspects described herein.

Phenyl: A cyclic group having skeletal formula of C6H5. In some aspects of the present disclosure, a phenyl group can have one or more groups (e.g., alkyl chains and/or halogen atoms) attached thereto.

Pulp Product: A product derived from a pretreated biomass according to the present disclosure and which is used as a feedstock to make a halomethylfurfural product according to a method described herein. In some aspects of the present disclosure, the pulp product is substantially free of hemicellulose (or any C5 sugars thereof) and/or lignin.

Six-Carbon (C6) Sugars: Sugars having a monomeric unit of 6 carbons and that may be of monomeric, dimeric, or polymeric form. The C6 sugars may include alcohol groups that are used in the conversion to halomethylfurfural. The C6 sugars can be cellulose and/or can be C6 sugars found in hemicellulose, both of which can be found in cellulosic biomass and lignocellulosic biomass. In some aspects of the present disclosure, the C6 sugars can be glucose, fructose, cellobiose, sucrose, lactose, maltose, or the like. In an independent aspect of the disclosure, methylated sugars (e.g., methyl-D-glucanopyranoside or dimethyl-4-O-methyl-hexanopyroside) may be included.

Solid Carbonaceous By-Product Material: A solid material that is produced during a method as described in U.S. Patent Publication No. 2014/0100378 and/or U.S. Pat. No. 9,388,151, wherein a biomass feedstock, or a pretreated pulp product, is converted to an HMF product. In particular aspects of the disclosure, the solid carbonaceous by-product material is isolated in an aqueous layer during the method.

Substantially Free: As used herein, the phrase “substantially free,” when used with the term “pulp product,” means that the pulp product contains a content of less than 50% (based on the total weight of the pulp product) of hemicellulose (or a C5 sugar thereof), lignin, or a combination thereof, such as less than 45%, less than 40%, less than 35%, less than 30%, less than 25%, less than 20%, less than 15%, less than 12%, less than 10%, less than 9%, less than 8%, less than 7%, less than 6%, less than 5%, less than 4%, less than 3%, less than 2%, and/or less than 1% (based on the total weight of the pulp product) of hemicellulose (or a C5 sugar thereof), lignin, or a combination thereof.

### Introduction

While separate independent processes exist in the art to (1) produce 5-(chloromethyl)furfural (“CMF”) from biomass (such as disclosed by U.S. Patent Publication No. 2014/0100378 and/or U.S. Pat. No. 9,388,151) and (2) pretreat biomass feedstocks to remove lignan (such as Kraft processing, Alcell processing, and/or sulfuric-acid catalyzed methanolysis), there exists a need for a process that can improve halomethylfurfural (“HMF”) yields, particularly CMF yields, while simultaneously decreasing production of undesired product. One example of such a byproduct is a solid carbonaceous by-product material produced during the process described by U.S. Patent Publication No. 2014/0100378 and/or U.S. Pat. No. 9,388,151. Production of this by-product can have the effect of reducing the yield of the HMF products produced in the method and also can increase the cost and complexity of the method.

Pretreating biomass using conventional processes, like Kraft processing, Alcell processing and/or sulfuric-acid catalyzed methanolysis, prior to converting the biomass to HMF products (e.g., CMF), may provide access to higher HMF yields (as compared to yields obtained from biomass that has not been pretreated); however, such increases may not always simultaneously result in a reduced amount of solid carbonaceous by-product material that is produced, at least to a level that would provide a desirable HMF: SCM ratio (wherein “SCM” represents the solid carbonaceous by-product material). Additionally, some conventional pretreatment processes can often require expensive and undesirable pretreatment components (e.g., expensive catalysts and/or strong acids). Additionally, certain conventional processes are not desirable for softwood biomass, such as Kraft processing, due to a lower content of hydrolysable ethereal α-O-4 and β-O-4 linkages.

The inventors of the present disclosure have determined how to increase HMF product yields while also reducing the amount of undesired byproducts produced in the method for converting a biomass feedstock to the HMF products described by U.S. Patent Publication No. 2014/0100378 and/or U.S. Pat. No. 9,388,151, particularly the solid carbonaceous by-product material formed during this method. While the present disclosure is not intended to be limited to a single pretreated pulp product used to make HMF (or any particular pretreatment process used to obtain such a pretreated pulp product), improved HMF yields and desirable HMF: SCM and/or HMF: by-product ratios (wherein HMF: by-product ratios represent the ratio of HMF to all by-products produced in the method, such by-products including the SCM, chlorinated by-products, and other by-products described herein) can be achieved using certain pretreatment processes described herein, including certain organosolv processes, and processes utilizing a treatment with a combination of a protic solvent and acetone. In representative aspects, a pretreatment process using a single-step acetone-water protocol according to aspects of the present disclosure provides desirable HMF (particularly CMF) yield improvement, as well as a significant reduction in byproduct formation, particularly a reduction in SCM amounts. In some aspects, the amount of such SCM can be reduced by at least 50% per unit biomass used in the method. For example, the amount of any SCM that might be formed when converting a pulp product to an HMF product can be reduced by at least 50% relative to an amount that would otherwise be formed without performing a pretreatment process disclosed herein.

In some aspects of the disclosure, the pretreatment process facilitates obtaining a pulp product that is substantially free of hemicellulose (including C5 sugars therein) and/or lignan, which increases the C6 sugar content per gram of pulp product feedstock. The disclosed method provides superior yields and product selectivity compared with conventional methods for converting biomass to useful products. Additionally, the disclosed method reduces by-product production and thus is useful for industrial applications where such compounds are not desired. Furthermore, pretreated materials are easier to transport through components of the apparatus or system used to make the HMF products (e.g., filters, pipes, etc.), which leads to a lower risk in facility design and higher potential ratios of biomass to water in reactors.

### Methods

The present disclosure describes a novel method that uses pretreated biomass to (i) increase the yield of halomethylfurfural (or “HMF”) products and (ii) reduce the amount of SCM, produced in a method wherein biomass is converted to HMF products, particularly using the method described by U.S. Patent Publication No. 2014/0100378 and/or U.S. Pat. No. 9,388,151. In particular aspects of the disclosure, the HMF product is CMF.

In some aspects of the present disclosure, a pulp product obtained from pretreated biomass is exposed to a mixture comprising a mineral acid, water, and an organic solvent at a reaction temperature sufficient to form HMF products, particularly CMF. In some aspects, the mixture can further comprise an inorganic salt component. Conditions disclosed in U.S. Patent Publication No. 2014/0100378 and/or U.S. Pat. No. 9,388,151 to convert biomass to the HMF products can be used in the method and the relevant portion of this method as disclosed by U.S. Patent Publication No. 2014/0100378 and/or U.S. Pat. No. 9,388,151 is incorporated herein by reference. In some aspects of the disclosure the pulp product is exposed to a mixture comprising a mineral acid, water, an organic solvent and an optional inorganic salt component at a reaction temperature ranging from 30° C. to 300° C., such as 30° C. to 275° C., or 30° C. to 250° C., or 30° C. to 225° C., or 30° C. to 220° C.

In some aspects of the present disclosure, the method further comprises the step of performing a pretreatment process on a biomass feedstock to produce the pulp product that is then exposed to the mixture comprising the mineral acid, water, and organic solvent to form the HMF products, particularly CMF. The pretreatment process can be a chemical pretreatment, a physical pretreatment, a physiochemical pretreatment, and/or a biological pretreatment, as described herein.

In some aspects of the disclosure, the pulp product is substantially free of hemicellulose (including C5 sugars therein) and/or lignin. In particular aspects of the disclosure, the pretreatment step used to produce the pulp product facilitates making a pulp product that is substantially free of hemicellulose (including C5 sugars therein) and/or lignin. In some aspects, reducing the amount of hemicellulose (including C5 sugars therein) and/or lignin facilitates increasing the C6 sugar content per gram of pulp product, thereby increasing the yield of HMF products. Without being limited to a single operating theory, it currently is believed that reducing the amount of hemicellulose (including C5 sugars therein) and/or lignin may facilitate reducing the amount of undesired by-products produced during the method whereby the pulp product is converted to HMF products, including the SCM that is isolated in the aqueous phase during HMF production.

In particular aspects of the disclosure, the biomass feedstock is lignocellulosic biomass feedstock. In other aspects, the biomass feed stock is a cellulosic biomass feedstock. However, the present disclosure is not limited to such examples and can include other biomass from other sources described herein. In yet other aspects, the biomass feedstock may comprise a saccharide composition, wherein the sugar composition includes a single saccharide or a mixture of saccharides (e.g., fructose, glucose, sucrose, lactose, and/or maltose). In particular aspects of the disclosure, the pulp product is obtained from pretreating a hardwood biomass feedstock or a softwood biomass feedstock (e.g., Southern Pine). In some aspects of the disclosure, the pulp product is obtained from treating a softwood biomass under organosolv conditions (e.g., sulfuric acid-catalyzed methanolysis), Alcell conditions (with or without acetone extraction, but preferably with acetone extraction), or a process utilizing a combination of a protic solvent and acetone, such as the single- or two-step water-acetone process described herein, with preferred aspects using a single-step water-acetone process.

In some aspects of the disclosure, the pretreatment process may comprise a chemical pretreatment, a physical pretreatment, a physiochemical pretreatment, and/or a biological pretreatment. In particular aspects of the disclosure, a chemical pretreatment process is performed on a biomass feedstock to produce a pulp product substantially free hemicellulose (including C5 sugars therein) and/or lignin, such that the pulp product includes a high amount of C6 sugars, including monomeric, dimeric, and/or polymeric C6 sugars comprising at least one alcohol group for the conversion into halomethylfurfural.

In some aspects, the pretreatment process is performed using a combination of a protic solvent and acetone and heat. In such aspects, the process is acid-free. In such aspects, the pretreatment process can be performed in a two-step manner or a single-step manner. In representative aspects, the protic solvent is water. In particular aspects, such as in a single-step method, the protic solvent and the acetone are used in amount that provide a 9:1 to 1:9 protic solvent: acetone ratio, or 9:1 to 1:8, or 9:1 to 1:7; or 9:1 to 1:6, or 9:1 to 1:5, or 9:1 to 1:4, or 9:1 to 1:3, or 9:1 to 1:2, or 9:1 to 1:1 protic solvent: acetone. In particular aspects, the ratio of protic solvent to acetone was 1:1.

In aspects utilizing a single-step protic solvent-acetone combination for pretreatment, the protic solvent and acetone are added to the biomass material, either as a single solution comprising a mixture of protic solvent and acetone, or as separate additions. After addition of the protic solvent and the acetone, the contents of the mixture are agitated and heated under pressure. In some aspects, the mixture is heated at temperatures ranging from 120° C. to 250° C., or 140° C. to 220° C., or 155° C. to 195° C. In some aspects, the mixture is heated under pressures using ballast gas (e.g., N2) ranging from 0 psig to 3000 psig, or 10 psig to 1500 psig, or 100 psig to 1000 psig, After a suitable time period, the reaction mixture is cooled and subject to drying, separation, and/or other purification techniques.

In aspects utilizing a two-step protic solvent-acetone combination for pretreatment, the biomass is first exposed to the protic solvent at elevated temperature and pressure for a suitable time. In some aspects, the mixture is heated at a temperature ranging from 120° C. to 250° C., or 140° C. to 220° C., or 155° C. to 195° C. Then, acetone is added (typically after removing volatile contents from the water-containing mixture) and the resulting mixture is heated at a temperature ranging from 50° C. to 120° C. After a suitable time period, the reaction mixture is cooled and subject to drying, separation, and/or other purification techniques.

In other aspects, the pretreatment process is an organosolv process, such as an organosolv process using sulfuric acid-catalyzed methanolysis. In particular aspects, the biomass feedstock is exposed to a solvent-containing reaction mixture comprising water, an organic pretreatment solvent, and a co-catalyst at an elevated temperature. In some aspects, the organic pretreatment solvent is selected from an alcohol, a carbonyl-containing solvent, an aldehyde-containing solvent, a glycol-containing solvent, or any combination thereof. In particular aspects of the disclosure, the organic pretreatment solvent is selected from methanol, ethanol, acetone, butanol, propanol, isopropyl alcohol, aldehyde-containing solvents, glycols, and the like, or any combination thereof. In particular aspects of the disclosure, the solvent is a mixture of acetone and methanol. In particular aspects, the co-catalyst comprises an acid selected from a mineral acid, an organic acid, or any combination thereof. In some aspects, the acid can be sulfuric acid, phosphoric acid, hydrochloric acid, formic acid, acetic acid, or any combination thereof. In some aspects, the elevated temperature ranges from 100° C. to 300° C., such as from 140° C. to 250° C., or 150° C. to 250° C., or 160° C. to 250° C. In some aspects, the sulfuric acid-catalyzed methanolysis pretreatment can be carried out at pressures ranging from 0 psig to 600 psig under an inert gas. In some aspects, the solubilization percent of the biomass feedstock can vary in the sulfuric acid-catalyzed methanolysis process. For example, in some aspects, greater than 0% to 60% of the starting mass of the biomass feedstock can be removed in the process. In particular aspects, 30% to 50% of the starting mass of the biomass feedstock can be removed in the process.

In other aspects, the pretreatment process is an Alcell process and in some particular aspects is an Alcell process that is combined with an acetone extraction step. In particular aspects, the Alcell process comprises using a mixture of MeOH and H₂O at a 4:1 ratio and heating at 190° C. In aspects using an acetone extraction step, the process is followed with an acetone extraction.

In some independent aspects, physical pretreatment can be used and involves using mechanical means for size reduction; gamma rays for radial formation and the breakdown of crystalline regions; microwaves for the disruption of lignin rupture hydrogen bonds; and/or ultrasound for oxidizing radical formation. Physiochemical pretreatment also may be used and can comprise using hydrothermal/liquid hot water techniques to form acid species; steam explosion for mechanical fracture and hydrolysis; and/or mechano-chemical applications for to reduce crystallinity due to mechanical forces. Biological pretreatments can be used and comprise using an enzyme, a microorganism, and/or a mechano-enzymatic technique to selectively degrade a substrate. In particular independent aspects, chemical pretreatments other than the organosolv, protic solvent-acetone combination process, and/or the Alcell process described above can be used. In such aspects, the chemical pretreatment can comprise using an alkaline treatment to breakdown lignin polysaccharide bonds (e.g., a Kraft processes); an acid treatment to degrade and solubilize hemicellulose; an ionic liquid treatment to dissolve cellulose; and/or a deep eutectic solvent treatment to dissolve lignin and hemicellulose.

In some independent aspects, a chemical process like Kraft pulping can be used. In such aspects, the biomass feedstock is exposed to a pulping mixture comprising water, sodium hydroxide, and sodium sulfide. The biomass typically is exposed to the pulping mixture at an elevated temperature. In particular aspects of the present disclosure, the elevated temperature can have range from 50° C. to 250° C.

In some independent aspects, a polar organic pretreatment solvent-based process can be used. Such a process can be used to separate lignin (in its oligomeric form) and methylated C5 sugars, and thus produces a pulp product comprising primarily C6 sugars. In particular aspects of the present disclosure, the polar organic solvent-based pretreatment comprises exposing the biomass feedstock to a polar organic pretreatment solvent in the presence of an inorganic acid and an added gas. In some aspects, the polar organic pretreatment solvent is methanol, ethanol, butanol, or any combination thereof. In other aspects, the inorganic acid is sulfuric acid, hydrochloric acid, phosphoric acid, or any combination thereof; and the added gas is nitrogen, air, hydrogen, or any combination thereof. In particular disclosed aspects, this pretreatment process comprises using an operating temperature ranging from 140° C. to 200° C., at normal pressure. In some aspects, the biomass feedstock is exposed to an operating temperature range of from 100° C. to 210° C. and at an operating pressure lower than 200 bar and at least 1 bar above the vapor pressure of the polar organic solvent at the operating temperature. In the polar organic solvent-based pretreatment process, the amount of water present is typically below 10 wt. %. In some aspects, the biomass feedstock is exposed for up to 300 minutes, 280 minutes, 270 minutes, 260 minutes. 250 minutes, 240 minutes, 230 minutes, 220 minutes, 210 minutes, 200 minutes.

In some aspects, the biomass feedstock comprises C6 sugars, C5 sugars, and lignin and the pulp product consists essentially of the C6 sugars from the biomass feedstock. In some other aspects, the pulp product can consist essentially of C6 sugars and C5 sugars other than C5 sugars in hemicellulose. In such aspects, the pulp product typically is free of components that would deleteriously affect converting the pulp product to an HMF product, and/or that would decrease the HMF product yield, and/or that would increase any SCM yield. For example, such components can include hemicellulose (including C5 sugars therein), lignin, and biomolecules other than cellulose that might be present in the biomass feedstock.

In particular disclosed aspects of the present disclosure, the HMF products are produced from the pulp product using a process as described in U.S. Patent Publication No. 2014/0100378 and/or U.S. Pat. No. 9,388,151, the relevant portion of which is hereby incorporated by reference. In particular aspects, converting the pulp product to the HMF products (including CMF) involves an acid-catalyzed conversion and uses organic solvents with temperature-dependent solubility for HMF products, and thus allows for temperature dependent phase separation of the HMF products from a reaction mixture.

In some aspects, the HMF product is produced by exposing the pulp product to a mixture comprising a mineral acid, water, and an organic solvent at a reaction temperature ranging from 30° C. to 300° C., such as 40° C. to 300° C., 50° C. to 250° C., 60° C. to 70° C., 80° C. to 100° C., 90° C. to 100° C., or 100° C. to 175° C. In some aspects, the reaction temperature is at least 30° C., at least 40° C., at least 50° C., at least 60° C., at least 70° C., at least 80° C., at least 90° C., at least 100° C., at least 150° C., at least 175° C., at least 200° C., or at least 250° C.

In particular disclosed aspects, the reaction pressure ranges from 0.1 atm to 10 atm. In other embodiments, the reaction pressure is atmospheric pressure.

In particular disclosed aspects, the mineral acid can be selected from hydrochloric acid, nitric acid, sulfuric acid, phosphoric acid, boric acid, hydrofluoric acid, hydrobromic acid, or any combination thereof. In an independent embodiment, other acids, like Lewis acids (e.g., AICI3 and FeCl3) can be used.

In some aspects, the organic solvent is a chemical compound comprising at least one alkyl group and at least one phenyl group wherein the alkyl group is attached to the phenyl group. For example, in particular disclosed aspects, the organic solvent can be an alkyl phenyl solvent, which may also be referred to as alkylbenzene or a phenylalkane, such as (monoalkyl)benzenes, (dialkyl)benzenes, and (polyalkyl)benzenes. In one exemplary aspect of the present disclosure, the organic solvent is toluene. In some aspects, the phenyl ring can be substituted with one or more halogen atoms. In certain embodiments, the organic solvent can be an alkyl(halobenzene). For example, the alkyl(halobenzene) may include alkyl(chlorobenzene). In one embodiment, the halo substituent for the phenyl ring may be, for example, chloro, bromo, or any combination thereof. In particular aspects of the disclosure, the organic solvent and the water provide a biphasic mixture that facilitates forming and/or isolating the HMF products.

In some aspects, converting the pulp product to the HMF product can further comprise using an inorganic salt component in combination with the mineral acid, water, and organic solvent. In such aspects of the present disclosure, the inorganic salt component can be selected from sodium chloride, magnesium chloride, calcium chloride, aluminum chloride, iron chloride, sodium bicarbonate, potassium chloride, sodium sulfate, calcium carbonate, calcium phosphate, or any combination thereof.

In certain aspects of the present disclosure, the HMF product is a solid 5-(halomethyl) furfural and can be amorphous or crystalline. In particular disclosed aspects of the present disclosure, the 5-(halomethyl) furfural product is 5-(chloromethyl)furfural (or “CMF”). In some aspects, the 5-(halomethyl) furfural is solid CMF. The solid CMF may be crystalline or amorphous. In other aspects, the 5-(halomethyl) furfural product is 5-(bromomethyl) furfural (BMF). In certain aspects, the 5-(halomethyl) furfural is solid BMF. The solid BMF may be crystalline or amorphous.

The HMF product produced according to the methods described herein may be used in other chemical reactions, or further downstream processing and converted into other furanic derivatives for biofuels, diesel additives, or plastics. For example, CMF may be converted into dimethylfuran and ethoxymethylfurfural.

The method can also comprise isolating the HMF product. Methods for isolating HMF are disclosed in U.S. Patent Publication No. 2014/0100378 and/or U.S. Pat. No. 9,388,151, the relevant portion of which is hereby incorporated by reference.

Pulp products made according to pretreatment processes of the present disclosure can provide product mixtures (after CMF production) wherein improved ratios of CMF to SCM are obtained, with particular pretreatment processes providing desirable CMF:SCM ratios while also meeting certain processing advantages, such as the ability to pretreat biomass without expensive or complex reagents and/or processing parameters. In some aspects, the CMF:SCM ratio may range from 0.4 to 2.5, such as 0.4 to 1.5, or 0.4 to 1, or 0.4 to 0.85, or 0.4 to 0.75. In some aspects, the CMF:SCM ratio ranges from 0.4 to 0.85, with particular examples providing ratios of 0.4, 0.51, 0.62, 0.71, 0.78, or 0.82. In some aspects, the pulp products also can provide product mixtures (after CMF production) wherein improved CMF to total by-product (represented a “ₜby-product”) ratios are obtained, with particular pretreatment processes providing desirable CMF: ₜby-product ratios while also meeting certain processing advantages, such as the ability to pretreat biomass without expensive or complex reagents and/or processing parameters. In some aspects, the CMF: ₜby-product ratio may range from 0.3 to 1.4, such as 0.3 to 0.8, or 0.3 to 0.6, or 0.3 to 0.5. In some aspects, the CMF: ₜby-product ratio ranges from 0.3 to 0.5, with particular examples providing ratios of 0.3, 0.33, 0.37, 0.41, 0.46, or 0.47. In particular examples, the pretreatment process used a single-step protic acid-acetone treatment, which provided a CMF:SCM ratio of 0.71 and a CMF: ₜby-product ratio of 0.41. While, in some aspects, other pretreatment processes may provide higher CMF:SCM and CMF: ₜby-product ratios than the single-step protic acid-acetone treatment, such as Kraft processing and/or organosolv processes described herein, these processes often use harsh conditions and/or require expensive and/or complex reagents that can have a disadvantageous effect on the implementation of such processes on an industrial scale. Additionally, pulp products obtained from Kraft processing may carry a higher sulfur content and/or require extensive filtration times than are desirable for HMF production methods.

In some aspects, the pretreatment process provides higher HMF yields based on mole percent of HMF produced. Higher HMF mol % can be an indication that more efficient utilization of the sugars used to produce CMF, rather than such sugars being converted to undesired by-products. In some aspects wherein the method produces CMF, the CMF mol % yield ranges from 40 mol % to 70 mol %, such as 40 mol % to 65 mol %, or 40 mol % to 60 mol %, or 40 mol % to 55 mol %. In particular aspects, CMF mol % yield was increased for particular pretreatment processes, such as organosolv processes, whereas similar increases were not observed for other processes, like Kraft pulping.

### Overview of Several Embodiments

A method according to aspects of the disclosure is described. In some aspects, the method comprises: combining (i) a pulp product obtained from pretreating a biomass feedstock with (ii) a first reaction mixture comprising, a mineral acid, water, and an organic solvent to form a second reaction mixture; and heating the second reaction mixture a reaction temperature ranging from 30° C. to 300° C. to produce a halomethylfurfural product-containing composition; and isolating the halomethylfurfural product from the halomethylfurfural product-containing composition.

In any or all aspects, the biomass feedstock comprises C6 sugars, C5 sugars, and lignin and the pulp product consists essentially of the C6 sugars from the biomass feedstock.

In any or all aspects, the reaction temperature has a range of 100° C. to 175° C.

In any or all aspects, the method further comprises combining the pulp product, the first reaction mixture, and/or the second reaction mixture with an inorganic salt.

In any or all aspects, the inorganic salt component is sodium chloride, magnesium chloride, aluminum chloride, iron chloride, calcium chloride, sodium bicarbonate, potassium chloride, sodium sulfate, calcium carbonate, calcium phosphate, or any combination thereof; the mineral acid is hydrochloric acid, nitric acid, sulfuric acid, phosphoric acid, boric acid, hydrofluoric acid, hydrobromic acid, or any combination thereof; and/or the organic solvent is a compound comprising at least one alkyl group and at least one phenyl group.

In any or all aspects, the pulp product is obtained from pretreating a softwood material using a chemical pretreatment process.

In any or all aspects, the chemical pretreatment process comprises exposing a mixture comprising the biomass feedstock to a protic solvent and acetone, adding a ballast gas at a pressure ranging from 0 psig to 1000 psig, and heating the mixture at a temperature ranging from 120° C. to 250° C.

In any or all aspects, the protic solvent and acetone are added to the mixture sequentially or simultaneously before heating.

In any or all aspects, the protic solvent is water.

In any or all aspects, the chemical pretreatment process comprises exposing the biomass feedstock to (i) an acid selected from a mineral acid, an organic acid, or any combination thereof, and (ii) a solvent-containing mixture comprising water and an organic pretreatment solvent selected from an alcohol, a carbonyl-containing solvent, an aldehyde-containing solvent, a glycol-containing solvent, or any combination thereof at a temperature ranging from 140° C. to 250° C.

In any or all aspects, the organic pretreatment solvent is selected from methanol, ethanol, butanol, propanol, isopropyl alcohol, acetone, or any combination thereof and the acid is selected from sulfuric acid, phosphoric acid, hydrochloric acid, formic acid, acetic acid, or any combination thereof.

In any or all aspects, the mineral acid is hydrochloric acid and the organic solvent is toluene.

In any or all aspects, the halomethylfurfural is 5-(chloromethyl)furfural.

In any or all aspects, the halomethylfurfural product-containing composition comprises an amount of solid carbonaceous by-product material.

In any or all aspects, using the pulp product in the method results in producing an amount of the solid carbonaceous by-product material that is lower than an amount of the solid carbonaceous by-product material produced by the method when using a biomass feedstock that is not pretreated.

In any or all aspects, the amount of the solid carbonaceous by-product material in the halomethylfurfural product-containing composition is reduced by 50% or more relative to the amount formed when the biomass feedstock is not pretreated.

In some aspects, the method comprises: performing a pretreatment process on a biomass feedstock to produce a pulp product that is substantially free of hemicellulose (including C5 sugars therein) and/or lignin; exposing the pulp product to a reaction mixture comprising, a mineral acid, water, and an organic solvent at a reaction temperature ranging from 30° C. to 300° C. to produce a halomethylfurfural product-containing composition; and isolating the halomethylfurfural product from the halomethylfurfural product-containing composition.

In any or all aspects, the biomass feedstock is a softwood.

In any or all aspects, the pretreatment process comprises a chemical treatment.

In any or all aspects, the chemical pretreatment process comprises exposing a mixture comprising the biomass feedstock to a protic solvent and acetone, adding a ballast gas at a pressure ranging from 0 psig to 1000 psig, and heating the mixture at a temperature ranging from 120° C. to 250° C.

In any or all aspects, the protic solvent and acetone are added to the mixture sequentially or simultaneously before heating.

In any or all aspects, the protic solvent is water.

In any or all aspects, the chemical pretreatment process comprises exposing the biomass feedstock to (i) an acid selected from a mineral acid, an organic acid, or any combination thereof, and (ii) a solvent-containing mixture comprising water and an organic pretreatment solvent selected from an alcohol, a carbonyl-containing solvent, an aldehyde-containing solvent, a glycol-containing solvent, or any combination thereof at a temperature ranging from 140° C. to 250° C.

In any or all aspects, the organic pretreatment solvent is selected from methanol, ethanol, butanol, propanol, isopropyl alcohol, acetone, or any combination thereof and the acid is selected from sulfuric acid, phosphoric acid, hydrochloric acid, formic acid, acetic acid, or any combination thereof.

In some aspects, the method comprises: performing a pretreatment process on a biomass feedstock, wherein the pretreatment process comprises exposing a mixture comprising the biomass feedstock to a protic solvent and acetone, adding a ballast gas at a pressure ranging from 0 psig to 1000 psig, and heating the mixture at a temperature ranging from 120° C. to 250° C. to produce a pulp product that is substantially free of hemicellulose (including C5 sugars therein) and/or lignin; exposing the pulp product to a reaction mixture comprising hydrochloric acid, water, and toluene at a reaction temperature ranging from 100° C. to 175° C. to produce a 5-(chloromethyl)furfural product-containing composition; and isolating 5-(chloromethyl)furfural from the 5-(chloromethyl)furfural product-containing composition; wherein any water present during the pretreatment process is present at an amount below 10 wt. %.

In any or all aspects, the protic solvent and acetone are added to the mixture sequentially or simultaneously before heating.

In any or all aspects, the protic solvent is water.

Also disclosed is a composition, comprising a halomethylfurfural product made according to any or all of the above method aspects.

In any or all aspects, the halomethylfurfural product is 5-(chloromethyl)furfural.

### EXAMPLES

### Example 1

In this example, a two-step acetone and water pretreatment process was performed on a softwood biomass feedstock. A 1 L Parr reactor was loaded with Southern Pine (70.0 g with 9.6% moisture content, or 63.28 g dry basis). DI water (361.9 g) was added, the reactor was sealed, 300 rpm agitation started, and ballast nitrogen gas pressure of 300 psi was added. The reactor was heated to 190° C. with pressure increasing to 617 psi and the temperature maintained for 90 minutes before cooling to ambient temperature. The contents of the reactor were transferred into a 1 L round bottom flask and the volatile components were removed in vacuo at 65° C. The resulting weight was 100.28 g with 53.3 g dry basis of material (16 wt % loss from original). A portion of this material (12.84 g, or 6.83 g dry basis, equivalent to 8.10 g of original wood dry basis) was placed into Soxhlet extractor and refluxed with 205 mL acetone overnight. The pulp was collected, briefly air dried, and dried in vacuum oven to 5.26 g (35.1 wt % overall removal). A dark red acetone fraction was evaporated to 1.67 g of dark red oil mixed with some grey solids (21 wt % of the original feedstock weight, possibly with moisture remaining).

### Example 2

In this example, a one-step acetone and water pretreatment process was performed on a softwood biomass feedstock. A 1 L Parr reactor was loaded with Southern Pine (51.78 g with 9.6% moisture content, or 46.81 g dry basis). DI water-acetone mixture (150 mL each) was added, the reactor was sealed, 300 rpm agitation started, and ballast nitrogen gas pressure of 293 psi was added. The reactor was heated to 190° C. with pressure increasing to 748 psi and the temperature maintained for 60 minutes before cooling to ambient temperature. The contents of the reactor were filtered and washed with approximately 200 mL acetone. The pulp was vacuum dried to 29.23 g (5.3% moisture or 27.68 g dry basis with 41% wt removal). The filtrate was removed in vacuo at 60° C. to 13.8 g of viscous dark red residue.

### Example 3

In this example, biomass to CMF reactions were carried out in pressure relief vials and yields of CMF products and SCM products were determined. Reactions were carried out with 7.5 wt % feedstock (approx. 580 mg dry basis), a 5.00 mL solution of 5.5 M CaCl₂) and 2.1 M HCl, and 8.00 mL toluene at a 130° C. heating block temperature (122° C. internal temperature after an 8-minute ramp-up period). Yields were determined using either a partial work-up method (yields based on product concentration in either organic or aqueous phase after aliquot sampling) or full work-up (yields based on product concentration in either organic or aqueous phase after filtration and excessive washing) of the reaction mixtures. Known organic products, such as CMF and furfural (or “FF”), and known aqueous phase products, such as levulinic acid (or “LA”) and formic acid (or “FA”), were measured by Analytical on GC and HPLC, respectively, while other unidentified by-products (referred to herein as “ASM” when isolated from the aqueous phase; and “OSM” when isolated from the organic phase) were quantified by difference as described in the above referenced report.

In this example, for comparison purposes, other pulp products were evaluated, including Kraft pulps (purchased from Canfor Pulp in Vancouver, CA) and pulp products obtained from two different organosolv processes known in the art (sulfuric acid-catalyzed methanolysis, and the Alcell process), which used Southern Pine wood as the initial biomass feedstock. The treatment conditions and weight removal of the pulps are shown in Table 1. Some pulps were analyzed and exhibited increased C6 content and C6/lignin ratio.

TABLE 1
Treatment types, conditions, and weight % removal from wood.

| | | Weight | | |
|---|---|---|---|---|
| | | removal | C6 | C6/ |
| Treatment type | Conditions | (wt %) | (wt %) | lignin |
| Southern Pine #218 | As is | 0 | 55.5 | 1.7 |
| Unbleached Kraft | High T alkaline sulfide | N/A | 81.2 | 7.5 |
| Bleached Kraft | Above + alkaline bleach | N/A | 85.3 | 12.1 |
| Alcell-type | MeOH—H₂O 4:1 (wt), | 21 | 53.5 | 3.2 |
| | 190° C. overnight | | | |
| Alcell + acetone | Above + Soxhlet acetone | 37 | TBD | TBD |
| extraction | extraction, overnight | | | |
| Sulfuric acid | MeOH, 7 mM H₂SO₄, | 45 | 61.4 | 2.5 |
| catalyzed | 190° C., 600 psi N₂, | | | |
| methanolysis | 45 min | | | |
| Dual step | H₂O, 190° C., 300 psi | 35 | TBD | TBD |
| water-acetone | N₂, 90 min, then | | | |
| | Soxhlet acetone | | | |
| | extraction, overnight | | | |
| Single step | H₂O-acetone 1:1 (v/v), | 41 | TBD | TBD |
| water-acetone | 190° C., 300 psi N₂, | | | |
| | 90 min | | | |

The amount of acetone extractives, which are preceded by water and ethanol extractions and typically represent terpenes in softwoods was also evaluated. Surprisingly, it was found that the acetone extractives increased from 0.2 wt % in Southern Pine wood to (a) 3.5 wt % in pulp obtained from the sulfuric acid-catalyzed methanolysis pretreatment and (b) 19.1 wt % in pulp obtained from the Alcell-type process. Without being limited to a single operating theory, it currently is believed that, while protic solvents can facilitate C—O bond cleavage in lignin, these solvents are not as efficient in lignin monomer extraction while also potentially being problematic due to partial hemicellulose/cellulose solvolysis and extraction of the CMF-producing C6 sugars. It was determined that combining a protic solvent with acetone could improve partial lignin depolymerization and extraction of the resulting fragments. Indeed, as shown in the last two entries of Table 1, there is more weight removal from wood using either two- or single-step water-acetone organosolv pretreatment (see Examples 1 and 2, respectively) compared to Alcell-type process.

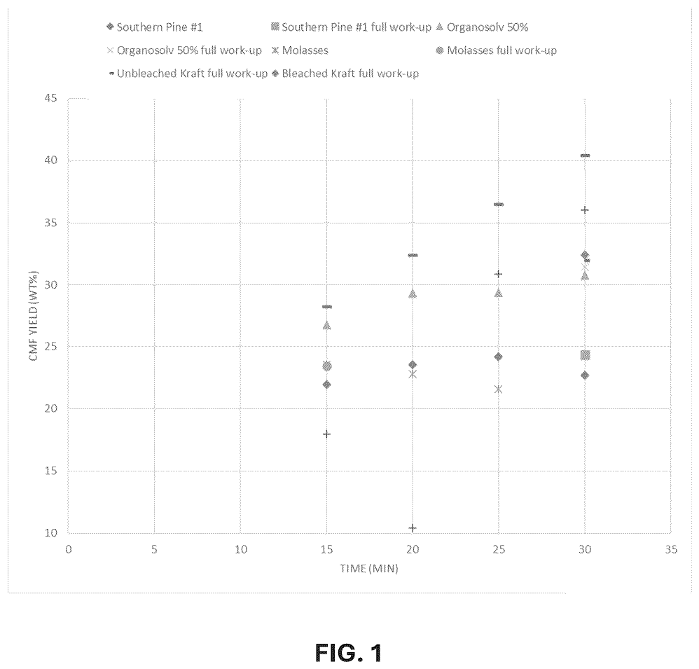

The resulting pulps from Table 1 were tested in pressure relief vials all at the same 7.5 wt % loading to determine product yields in the biomass to CMF reaction. CMF and solid carbonaceous by-product material yields are shown in Table 2. FIG. 1 shows a graph of results for CMF yield (wt %) over the course of reaction time. CMF yields from all the pulps are higher than from the original wood due to increased sugar weight content relative to the original wood sample. Consistent with high lignin removal, Kraft pulps produced the lowest solid carbonaceous by-product material yield. Filtration of Kraft-based solid carbonaceous by-product material was extremely slow in the case of the bleached pulp. Additionally, CMF yield was lower with full work-up method compared to partial work-up. This difference was 4 wt % for the unbleached pulp and 8 wt % with the bleached Kraft pulp. These drops in yield between the two methods may indicate that part of the organic phase was lost, perhaps due to entrapment inside of a highly cross-linked solid carbonaceous by-product material that lacks the rigid structure of wood.

TABLE 2
CMF in HTC yields from pulps in Table 1 in pressure
relief vials based on full work-up method under standard
conditions. Error of analysis is ±5% relative.

| | CMF yield | | HTC yield | |
|---|---|---|---|---|
| | % pulp | % wood | % pulp | % wood |
| Treatment type | wt | wt | wt | wt |
| Southern Pine #1 | 24.3 | 23.6 | 39.5 | 41.1 |
| Unbleached Kraft | 32.0 | N/A | 20.1 | N/A |
| Bleached Kraft | 32.4 | N/A | 16.1 | N/A |
| Alcell + acetone extraction | 35.4 | 22.3 | 36.9 | 23.2 |
| Sulfuric acid catalyzed | 31.5 | 17.3 | 40.5 | 23.1 |
| methanolysis | | | | |
| Dual step water-acetone | 27.0 | 17.6 | 66.9 | 43.5 |
| Single step water-acetone | 29.1 | 17.2 | 40.8 | 24.1 |
| Southern Pine #2 | 21 | N/A | 41.1 | N/A |
| Alcell | 25.4 | N/A | N/A | N/A |
| Cornstarch | 49.1 | N/A | 7.22 | N/A |
| Corn Kernel | 42 | N/A | 13 | N/A |
| Soybean Molasses | 23.4 | N/A | 6.8 | N/A |

CMF yields based on the original wood weight dropped for all treated pulps due to overall losses of C6 sugars during the pretreatment process. The highest overall CMF yield was in case of acetone extracted Alcell-type pulp indicating that combining protic solvents with acetone could be a promising approach for integration with biomass to CMF conversion. This was also the pulp with lowest solid carbonaceous by-product material yield, following the Kraft pulps. The single-step, acid-free water-acetone pulp, provided CMF and solid carbonaceous by-product material yield was comparable to the pulp obtained from sulfuric acid-catalyzed methanolysis, without any optimization.

CMF yield based on C6 sugar content was noticeably higher for the pulp obtained from sulfuric acid-catalyzed methanolysis compared to the original Southern Pine feedstock (see FIG. 1). Without being limited to a single operating theory, it currently is believed that this may be due to an increase of glucose content among other less selective C6 sugars from 73% in Southern Pine to 96% in the pulp obtained from the sulfuric acid-catalyzed methanolysis. It also is believed that the sulfuric acid-catalyzed methanolysis process may be reducing the porosity of the woody biomass which facilitates mass transfer of CMF from the aqueous into the organic phase in the biomass to CMF reaction step.

Yields of the other by-products were unremarkable and comparable to that of the original wood when considering higher CMF yield and thus higher amount of decomposition to LA and FA (Table 3).

TABLE 3
By-product yields from pulps in Table 1 in pressure relief vials
based on full work-up method under standard conditions. Error of
analysis is ±5% relative, except for ASM and OSM with ±9% relative.

| | FF | FA | LA | ASM | OSM |
|---|---|---|---|---|---|
| Treatment type | (wt %) | (wt %) | (wt %) | (wt %) | (wt %) |
| Southern Pine #1 | 4.2 | 1.8 | 3.3 | 13 | 10 |
| Unbleached Kraft | 4.3 | 2.6 | 5.2 | 9 | 7 |
| Bleached Kraft | 3.3 | 2.4 | 6.2 | 9 | 9 |
| Alcell + acetone | NQ | 2.5 | 4.2 | 9 | NQ |
| extraction | | | | | |
| Sulfuric acid catalyzed | 1.7 | 2.6 | 4.3 | 6 | 9 |
| methanolysis | | | | | |
| Dual step water-acetone | NQ | 1.7 | 3.3 | 10 | NQ |
| Single step water-acetone | NQ | 2.5 | 4.3 | 16 | NQ |

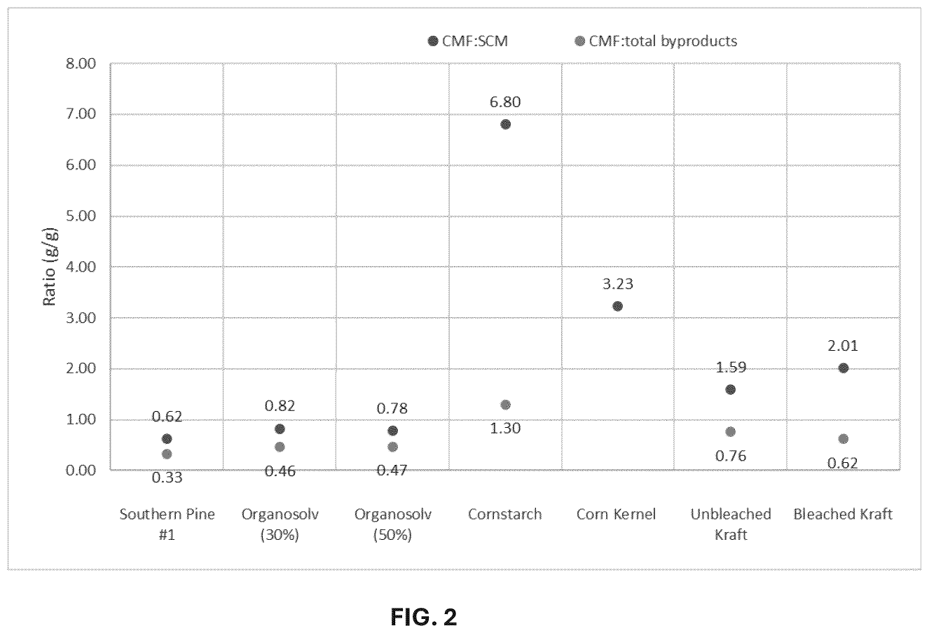

Additional parameters were measured, including those listed in Tables 4 and 5, below. With reference to Table 4, the column labeled “OS (0 psig)” provides results for a pretreatment process using sulfuric acid catalyzed methanolysis at 0 psig with 30% solubilization of the biomass (i.e., 30% of the starting mass was removed) and the column labeled “OS (600 psig)” provides results for a pretreatment process using sulfuric acid catalyzed methanolysis at 600 psig with 50% solubilization of the biomass (i.e., 50% of the starting mass was removed). CMF-to-SCM ratios and CMF: ₜby-product ratios are depicted graphically in FIG. 2.

TABLE 4
Additional measured parameters for different pretreated pulp products

| | | | | | | 1 step | 1 step |
|---|---|---|---|---|---|---|---|
| | Southern | OS (30%; | OS (50%, | Unbleached | Bleached | H₂O- | H₂O- |
| | Pine #1 | 0 psig) | 600 psig) | Kraft | Kraft | acetone | acetone |
| CMF Yield (wt %) | 24.3 | 30.7 | 31.5 | 32.0 | 32.4 | 29.1 | 27 |
| CMF Molar Yield | 47.5 | 54.5 | 56.8 | 44.13 | 42.58 | | |
| C6:Lignin | 1.7 | 2.4 | 2.5 | 7.5 | 12.1 | | |
| C6:(Lignin + | 1.5 | 1.8 | 1.8 | 6.8 | 10.1 | | |
| Extractives) | | | | | | | |
| C6:Everything | 1.2 | 1.6 | 1.7 | 4.0 | 5.0 | | |
| Else | | | | | | | |
| SCM Yield (wt %) | 39.5 | 37.4 | 40.5 | 20.1 | 16.1 | 40.8 | 66.9 |
| Non SCM/CMF | 34.0 | 29.6 | 26.6 | 22.0 | 36.5 | 30.1 | 6.1 |
| Products (wt %) | | | | | | | |
| OSM Yield (Dry | 10.3 | 8.6 | 8.5 | 6.6 | 9.3 | | |
| Mass Basis) | | | | | | | |
| wt % Lignin | 32.64 | 25.51 | 25.02 | 10.8 | 7.1 | | |
| Total Byproduct | 73.5 | 67.0 | 67.1 | 42.2 | 52.6 | 70.9 | 73.0 |
| Yield | | | | | | | |
| Non C6 | 44.74 | 39.06 | 37.09 | 20.79 | 17.54 | | |
| Composition | | | | | | | |
| Product Mass | 91.3 | 98.5 | 97.8 | 74.1 | 85.0 | | |
| Balance | | | | | | | |
| CMF:SCM | 0.62 | 0.82 | 0.78 | 1.59 | 2.01 | 0.71 | 0.40 |
| CMF:ₜbyproducts | 0.33 | 0.46 | 0.47 | 0.76 | 0.62 | 0.41 | 0.37 |

TABLE 5
Additional measured parameters for
different pretreated pulp products

| | Southern | Alcell- | Corn- | Corn | Soybean |
|---|---|---|---|---|---|
| | Pine #2 | Type | starch | Kernel | Molasses |
| CMF Yield (wt %) | 21.0 | 25.4 | 49.1 | 42 | 23.4 |
| CMF Molar Yield | 44.7 | 43.1 | 57.0 | 63.0 | — |
| C6:Lignin | 1.6 | 3.2 | — | — | 7.1 |
| C6:(Lignin + | 1.4 | 1.3 | N/A | — | — |
| Extractives) | | | | | |
| C6:Everything Else | 1.1 | 1.2 | N/A | — | 1.8 |
| SCM Yield (wt %) | 41.1 | — | 7.22 | 13.0 | 6.8 |
| Non SCM/CMF | 29.7 | N/A | 30.5 | — | — |
| Products (wt %) | | | | | |
| OSM Yield (Dry | 6.6 | N/A | 6 | — | N/A |
| Mass Basis) | | | | | |
| wt % Lignin | 31.78 | 17.33 | N/A | — | — |
| Total Byproduct | 70.8 | N/A | 37.7 | — | N/A |
| Yield | | | | | |
| Non C6 | 46.59 | 46.43 | N/A | — | — |
| Composition | | | | | |
| Product Mass | 92.0 | N/A | 86.9 | — | — |
| Balance | | | | | |
| CMF:SCM | 0.51 | — | 6.80 | 3.23 | — |
| CMF:ₜbyproducts | 0.30 | — | 1.30 | — | — |

In view of the many possible embodiments to which the principles of the present disclosure may be applied, it should be recognized that the illustrated embodiments are only preferred examples and should not be taken as limiting the scope of the present disclosure. Rather, the scope is defined by the following claims. We therefore claim as our invention all that comes within the scope and spirit of these claims.

We claim:

1. A method, comprising:
combining (i) a pulp product obtained from pretreating a biomass feedstock that is a softwood material using a chemical pretreatment process with (ii) a first reaction mixture comprising, a mineral acid, water, and an organic solvent to form a second reaction mixture;
heating the second reaction mixture at a reaction temperature ranging from 30° C. to 300° C. to produce a halomethylfurfural product-containing composition; and
isolating a halomethylfurfural product from the halomethylfurfural product-containing composition.

2. The method of claim 1, wherein the biomass feedstock comprises C6 sugars, C5 sugars, and lignin and the pulp product consists essentially of the C6 sugars from the biomass feedstock.

3. The method of claim 1, wherein the reaction temperature has a range of 100° C. to 175° C.

4. The method of claim 1, further comprising combining the pulp product, the first reaction mixture, and/or the second reaction mixture with an inorganic salt.

5. The method of claim 4, wherein the inorganic salt is sodium chloride, magnesium chloride, aluminum chloride, iron chloride, calcium chloride, sodium bicarbonate, potassium chloride, sodium sulfate, calcium carbonate, calcium phosphate, or any combination thereof; the mineral acid is hydrochloric acid, nitric acid, sulfuric acid, phosphoric acid, boric acid, hydrofluoric acid, hydrobromic acid, or any combination thereof; and/or the organic solvent is a compound comprising at least one alkyl group and at least one phenyl group.

6. The method of claim 1, wherein the chemical pretreatment process comprises exposing a mixture comprising the biomass feedstock to a protic solvent and acetone, adding a ballast gas at a pressure ranging from 0 psig to 1000 psig, and heating the mixture at a temperature ranging from 120° C. to 250° C.

7. The method of claim 6, wherein the protic solvent and acetone are added to the mixture sequentially or simultaneously before heating.

8. The method of claim 6, wherein the protic solvent is water.

9. The method of claim 1, wherein the chemical pretreatment process comprises exposing the biomass feedstock to (i) an acid selected from a mineral acid, an organic acid, or any combination thereof, and (ii) a solvent-containing mixture comprising water and an organic pretreatment solvent selected from an alcohol, a carbonyl-containing solvent, an aldehyde-containing solvent, a glycol-containing solvent, or any combination thereof at a temperature ranging from 140° C. to 250° C.

10. The method of claim 9, wherein the organic pretreatment solvent is selected from methanol, ethanol, butanol, propanol, isopropyl alcohol, acetone, or any combination thereof and the acid is selected from sulfuric acid, phosphoric acid, hydrochloric acid, formic acid, acetic acid, or any combination thereof.

11. The method of claim 1, wherein the mineral acid is hydrochloric acid and the organic solvent is toluene.

12. The method of claim 1, wherein the halomethylfurfural product is 5-(chloromethyl)furfural.

13. The method of claim 1, wherein the halomethylfurfural product-containing composition comprises an amount of solid carbonaceous by-product material.

14. The method of claim 13, wherein using the pulp product in the method results in producing an amount of the solid carbonaceous by-product material that is lower than an amount of the solid carbonaceous by-product material produced by the method when using a biomass feedstock that is not pretreated.

15. The method of claim 14, wherein the amount of the solid carbonaceous by-product material in the halomethylfurfural product-containing composition is reduced by 50% or more relative to the amount formed when the biomass feedstock is not pretreated.

16. A method, comprising:
performing a pretreatment process on a biomass feedstock that is a softwood to produce a pulp product that is substantially free of hemicellulose (including C5 sugars therein) and/or lignin;
exposing the pulp product to a reaction mixture comprising, a mineral acid, water, and an organic solvent at a reaction temperature ranging from 30° C. to 300° C. to produce a halomethylfurfural product-containing composition; and
isolating a halomethylfurfural product from the halomethylfurfural product-containing composition.

17. The method of claim 16, wherein the pretreatment process comprises a chemical treatment.

18. The method of claim 17, wherein the chemical pretreatment process comprises exposing a mixture comprising the biomass feedstock to a protic solvent and acetone, adding a ballast gas at a pressure ranging from 0 psig to 1000 psig, and heating the mixture at a temperature ranging from 120° C. to 250° C.

19. The method of claim 18, wherein the protic solvent and acetone are added to the mixture sequentially or simultaneously before heating.

20. The method of claim 18, wherein the protic solvent is water.

21. The method of claim 17, wherein the chemical pretreatment process comprises exposing the biomass feedstock to (i) an acid selected from a mineral acid, an organic acid, or any combination thereof, and (ii) a solvent-containing mixture comprising water and an organic pretreatment solvent selected from an alcohol, a carbonyl-containing solvent, an aldehyde-containing solvent, a glycol-containing solvent, or any combination thereof at a temperature ranging from 140° C. to 250° C.

22. The method of claim 21, wherein the organic pretreatment solvent is selected from methanol, ethanol, butanol, propanol, isopropyl alcohol, acetone, or any combination thereof and the acid is selected from sulfuric acid, phosphoric acid, hydrochloric acid, formic acid, acetic acid, or any combination thereof.

23. A method, comprising:
performing a pretreatment process on a biomass feedstock, wherein the pretreatment process comprises exposing a mixture comprising the biomass feedstock to a protic solvent and acetone, adding a ballast gas at a pressure ranging from 0 psig to 1000 psig, and heating the mixture at a temperature ranging from 120° C. to 250° C. to produce a pulp product that is substantially free of hemicellulose (including C5 sugars therein) and/or lignin;
exposing the pulp product to a reaction mixture comprising hydrochloric acid, water, and toluene at a reaction temperature ranging from 100° C. to 175° C. to produce a 5-(chloromethyl)furfural product-containing composition; and
isolating 5-(chloromethyl)furfural from the 5-(chloromethyl)furfural product-containing composition; wherein any water present during the pretreatment process is present at an amount below 10 wt. %.

24. The method of claim 23, wherein the protic solvent and acetone are added to the mixture sequentially or simultaneously before heating.

25. The method of claim 23, wherein the protic solvent is water.

26. A composition, comprising a halomethylfurfural product made according to the method of claim 1.

27. The composition of claim 26, wherein the halomethylfurfural product is 5-(chloromethyl)furfural.
